(19) United States
(12) Patent Application Publication
(10) Pub. No.: US 2018/0000932 A1
(43) Pub. Date: Jan. 4, 2018

(54) FORMULATION OF AGLYCOSYLATED THERAPEUTIC ANTIBODIES

(71) Applicant: NOVELMED THERAPEUTICS, INC., Cleveland, OH (US)

(72) Inventor: Rekha BANSAL, Solon, OH (US)

(21) Appl. No.: 15/541,057

(22) PCT Filed: Dec. 31, 2015
(86) PCT No.: PCT/US15/68327

§ 371 Date: Jun. 30, 2017

Related U.S. Application Data
(60) Provisional application No. 62/098,491, filed on Dec. 31, 2014.

(51) Int. Cl. A61K 39/395; C07K 16/18

(52) U.S. Cl. CPC A61K 39/39591; C07K 16/18

(57) ABSTRACT

Described herein are antibody formulations including a therapeutic antibody at a concentration of at least 20 mg/ml, methods for optimizing and producing such antibody formulations, and methods of using such antibody formulations. Antibody formulations including a therapeutic antibody at a concentration of at least about 20 mg/mL are described herein. For example, described are high concentration solutions and formulations of aglycosylated antibod(ies), methods of making such formulations, and methods for using such formulations. The described formulations, when solutions, exhibit reduced viscosity and good stability.

### BACKGROUND

One limitation of engineered antibodies as therapeutic biologics is the difficulty in formulating stable solutions of concentrated antibody. At higher concentrations antibody molecules tend to aggregate and fall out of solution. Aglycosylation reduces repulsive forces between molecules and allows the antibodies to come into closer proximity to each other. Consequently, aglycosylation results in decreased stability and increased aggregation. Aglycosylation, however, does provide many therapeutic and pharmacologic advantages in vivo. Aglycosylated antibodies are also, typically, easier to manufacture. For these and other reasons, aglycosylated antibodies are a preferred form of therapeutic antibody.

The majority of therapeutic antibody formulations have been developed for the use of glycosylated antibodies in intravenous applications, which do not require high concentration formulations. The in vivo utility of aglycosylated antibodies creates a need to identify stable, high concentration aglycosylated antibody formulations suitable for therapeutic use.

### SUMMARY

Antibody formulations including a therapeutic antibody at a concentration of at least about 20 mg/mL are described herein. For example, described are high concentration solutions and formulations of aglycosylated antibod(ies), methods of making such formulations, and methods for using such formulations. The described formulations, when solutions, exhibit reduced viscosity and good stability. These high concentration formulations are easy to handle and are suitable for subcutaneous and intravitreal (IVT) administration, for example. These formulations are suitable, for example, for IVT administration of therapeutic antibodies for the treatment of ocular disorders, and for subcutaneous administration for acute or chronic systemic conditions. Also described herein are methods for reducing the viscosity of concentrated solutions of any aglycosylated antibody.

In one embodiment, the invention accordingly features a high concentration antibody formulation including a therapeutic antibody or a fragment thereof at a concentration of at least about 20 mg/mL. In a particular embodiment, the therapeutic antibody concentration is at least about 100 mg/mL to about 250 mg/mL or higher, e.g., about 160 mg/mL. In a particular embodiment the formulation is a solution with a viscosity of less than about 60 centipoise (cP), e.g., between about 20 cP and about 50 cP. In a particular embodiment, the solution has a viscosity of about 20 cP. In a particular embodiment, the formulations described herein further include a buffer selected from the group consisting of: histidine buffer, citrate buffer, acetate buffer, succinate buffer and phosphate buffer, e.g., a histidine buffer including a combination selected from the group of: histidine chloride and arginine chloride; histidine acetate and arginine acetate; histidine phosphate and arginine phosphate; histidine sulfate and arginine sulfate; and histidine succinate and arginine succinate.

In a particular embodiment, the formulations described herein further include a viscosity reducing agent selected from the group consisting of: arginine, glycine, lysine, histidine, glutamic acid, aspartic acid, isoleucine, leucine, alanine, phenylalanine, tyrosine, tryptophan, methionine, serine and proline.

In a particular embodiment, the formulations described herein further include an agent to prevent antibody aggregation, wherein the agent is selected from the group consisting of: arginine, glycine, lysine, histidine, glutamic acid, aspartic acid, isoleucine, leucine, alanine, phenylalanine, tyrosine, tryptophan, methionine, serine and proline.

In a particular embodiment, the pH of the formulation is between about 5.0 and about 6.5.

In a particular embodiment, the formulations described herein further include PS-80 at a concentration between about 0.005% and about 0.10%, e.g., at about 0.01% to about 0.04%.

In a particular embodiment, the formulations described herein further include a salt selected from the group consisting of: sodium chloride, sodium thiocyanate, ammonium thiocyanate, ammonium sulfate, ammonium chloride, calcium chloride, arginine hydrochloride, zinc chloride and sodium acetate. In a particular embodiment, the formulations described herein further include sodium chloride at a concentration between about 20 mM and about 150 mM or between about 150 mM and about 250 mM. In a particular embodiment, the formulations described herein further include a lyoprotectant. In a particular embodiment, the formulations described herein further include a sugar selected from the group consisting of: glucose, sucrose, trehalose, lactose, fructose, maltose, dextran, glycerin, dextran, erythritol, glycerol, arabitol, sylitol, sorbitol, mannitol, melibiose, melezitose, raffinose, mannotriose, stachyose, maltose, lactulose, maltulose, glucitol, maltitol, lactitol and isomaltulose. The sugar can be present, for example, in an amount of about 0.5% to about 5%.

In a particular embodiment, the formulations described herein further include a surfactant, e.g., a surfactant selected from the group consisting of: polysorbate 20; polysorbate 80 (PS-80); a poloxamer; poloxamer 188; Triton; sodium dodecyl sulfate (SDS); sodium laurel sulfate; sodium octyl glycoside; lauryl-, myristyl-, linoleyl-, and stearyl sulfobetaine; lauryl-, myristyl-, linoleyl- and stearyl sarcosine; linoleyl-, myristyl- and cetyl betaine; lauroamidopropyl-, cocamidopropyl-, linoleamidopropyl-, myristamidopropyl-, palmidopropyl- and isostearamidopropyl betaine; lauroamidopropyl; myristamidopropyl-, palmidopropyl- and isostearamidopropyl dimethylamine; sodium methyl cocoyl- and disodium methyl oleyl taurate; the MONAQUAT™ series; polyethyl glycol, polypropyl glycol, and copolymers of ethylene and propylene glycol; and Pluronic® block polymers.

In a particular embodiment, the therapeutic antibody is reconstituted from a lyophilized form, e.g., wherein the antibody concentration in the reconstituted formulation is approximately 2 to 40 times greater than the protein concentration of the antibody prior to lyophilization.

In a particular embodiment, the antibody is selected from the group consisting of: an aglycosylated antibody, a hybrid antibody of IgG1/IgG2, a hybrid antibody IgG2/IgG4, an IgG1 antibody, an IgG2 antibody and combinations or fragments thereof. In one embodiment, the antibody is an aglycosylated antibody.

In a particular embodiment, the antibody is selected from the group consisting of: an anti-properdin antibody, an anti-factor B antibody, an anti-C3 antibody, an anti-factor D antibody, an anti-factor C5 antibody, and an anti-C5b-9 antibody. In a particular embodiment, the formulation includes 25 mM histidine, 2% sucrose, 110 mM NaCl and 0.01% Tween at a pH about 6.5.

In one embodiment, the disclosure is directed to a method of administering a therapeutic antibody to a patient in need thereof including administering a therapeutically effective amount of a formulation described herein. In a particular embodiment, the formulation is administered subcutaneously, intravitreally or intravenously.

### BRIEF DESCRIPTION OF THE DRAWINGS

FIG. 1 shows the occurrence of High Molecular Weight (HMW) proteins in various combinations of excipients and buffers, and at various different pH levels. Data were collected using size exclusion chromatography, with formulations at 40° C. There are five data bars for each combination of pH and buffer. From left to right within each group, the data bars represent formulations with the following excipients added: none (control), 150 mM NaCl, 5% sucrose, 200 mM sorbitol, 5% PEG-200 (Poly Ethylene Glycol 200), 250 mM arginine sulfate.

FIG. 2 shows the occurrence of HMW proteins in various combinations of excipients and buffers, and at various pH levels. Data were collected using size exclusion chromatography with formulations at 5° C. There are five data bars for each combination of pH and buffer. From left to right within each group, the data bars represent formulations with the following excipients added: none (control), 150 mM NaCl, 5% sucrose, 200 mM sorbitol, 5% PEG-200, 250 mM ArgSO₄

FIG. 3 shows dynamic light scattering data measuring the average molecular diameter of the antibody as measured in formulations utilizing different excipients and buffers.

FIG. 4 shows evaluation of effect of various concentrations of NaCl on the pH of different formulations using citrate or histidine buffer.

FIG. 5 shows SEC (size exclusion chromatography) evaluation of effect of various concentrations of NaCl on different formulations using citrate or histidine buffer.

FIG. 6 shows that the stability of the aglycosylated antibody in the base formulation can be substantially improved with the addition of 0.01 to 0.02% PS-80.

FIG. 7 shows the relationship between viscosity and antibody concentration in 20 mM histidine, 150 mM NaCl, pH 6.0 without the addition of polysorbate. At a concentration of approximately 200 mg/mL, the viscosity of the solution is nearly 50 cp. Arginine could be added to lower the viscosity. For a 50 mg/mL formulation, viscosity values are expected to be nearly 2 cP without the addition of polysorbate. Data were generated using a formulation of a test antibody at various concentrations. The test antibody was an anti-complement factor antibody, e.g., an anti-properdin antibody.

FIG. 8 shows the results of using viscosity reducing agents in the high-concentration antibody formulations described herein. Results showing viscosity vs. shear rate are shown for a 50 mM histidine buffer, where the antibody concentration of the test antibody was 209 mg/mL and for a 100 mM histidine buffer, where the antibody concentration was 219 mg/mL. Measurements were taken at 22° C.

FIG. 9 shows viscosity vs. shear rate data for various high-concentration antibody formulations. Test antibody concentrations were fairly consistent for each formulation, ranging from 200 mg/mL to 219 mg/mL. Data were obtained at 22° C.

FIGS. 10A and 10B show viscosity vs. shear rate data measured at 22° C. for various high-concentration antibody formulations.

FIGS. 11A-11D show viscosity vs. shear rate data measured at 22° C. for various high-concentration antibody formulations.

FIG. 11E shows the relationship between viscosity and temperature (test antibody concentration was 162 mg/mL (20 mM histidine, 150 mM NaCl, 0.02% PS-80, pH=6.0)).

FIGS. 12A and 12B show viscosity vs. shear rate data measured at 22° C. for various high-concentration antibody formulations.

### DETAILED DESCRIPTION

Described herein are high-concentration antibody formulations and methods of manufacturing and using such formulations. Such high-concentration antibody formulations can include, for example, aglycosylated antibodies or other modified antibodies or fragments thereof. The high-concentration formulations described herein are useful, for example, for therapeutic delivery of antibodies to a subject in need thereof.

As used herein, “formulation” refers to a specific recipe for a composition, e.g., a solution, which uses a combination of, for example, diluents, buffers and other compounds. Formulations described herein can also include, for example, one or more of a bulking agent, an excipient, a surfactant, a preservative or a stabilizer.

As used herein, “diluent” refers to compounds to normalize the concentration of an active ingredient, e.g., an antibody or fragment thereof. Diluents that are suitable for use in the formulations described herein include, for example, pharmaceutically acceptable inert fillers such as microcrystalline cellulose, lactose, sucrose, fructose, glucose dextrose, or other sugars, dibasic calcium phosphate, calcium sulfate, cellulose, ethylcellulose, cellulose derivatives, kaolin, mannitol, lactitol, maltitol, xylitol, sorbitol, or other sugar alcohols, dry starch, saccharides, dextrin, maltodextrin or other polysaccharides, inositol or mixtures thereof. The diluent can be, for example, a water-soluble diluent. Examples of diluents include, for example: microcrystalline cellulose such as Avicel® PH112, Avicel® PH101 and Avicel® PH102; lactose such as lactose monohydrate, lactose anhydrous, and Pharmatose® DCL 21; dibasic calcium phosphate (e.g., Emcompress®); mannitol; starch; sorbitol; sucrose; and glucose. The diluent can be used in an amount of about 2% to about 80% by weight, about 20% to about 50% by weight, or about 25% by weight of the treatment formulation.

As used herein, a “buffer” is a solution that resists pH change upon the addition of acidic or basic components by neutralizing small amounts of added acid or base, thus maintaining the pH of the solution relatively stable. To effectively maintain a pH range, a buffer includes a weak conjugate acid-base pair (a weak acid and its conjugate base or a weak base and its conjugate acid).

As used herein, a “bulking agent” refers to a compound that adds mass to a lyophilized mixture and contributes to the physical structure of a lyophilized cake (e.g., facilitates the production of an essentially uniform lyophilized cake that maintains an open pore structure). Exemplary bulking agents include mannitol, glycine, polyethylene glycol and sorbitol. The liquid formulations described herein can be prepared, for example, by reconstitution of a lyophilized formulation including such bulking agents.

The active ingredient(s), such as antibodies or antibody fragments, of the formulations described herein can be mixed, for example, with excipients, which are pharmaceutically acceptable and compatible with the active ingredient and in amounts suitable for use in the therapeutic methods described herein. The term “excipient” refers to an agent that may be added to a preparation or formulation to provide some benefit to the quality and/or stability of the solution. For example, an excipient may be added to a solution of therapeutic antibody to act as a stabilizer, to achieve a desired consistency (e.g., altering bulk properties), and/or to adjust osmolality. Examples of excipients include, but are not limited to, stabilizers, sugars, polyols, amino acids, surfactants, chelating agents, and polymers. Various excipients can be homogeneously mixed with the active agent of the present disclosure as would be known to those skilled in the art. The active agent, for example, can be mixed or combined with excipients such as but not limited to microcrystalline cellulose, colloidal silicon dioxide, lactose, starch, sorbitol, cyclodextrin and combinations of these.

Other agents that can be used in the formulations and methods described herein include, for example, a surfactant. The term “surfactant” refers to a surface-active agent, preferably a nonionic surfactant. Examples of surfactants include, for example, polysorbate (e.g., polysorbate 20 and polysorbate 80); poloxamer (e.g., poloxamer 188); triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl- or isostearamidopropyl-betaine (e.g., lauroamidopropyl); myristamidopropyl-, palmidopropyl- or isostearamidopropyl-dimethylamine; sodium methyl cocoyl- or disodium methyl oleyl-taurate; and the MONAQUAT™ series (Mona Industries, Inc., Paterson, N.J.); polyethyl glycol, polypropyl glycol, and copolymers of ethylene and propylene glycol (e.g., Pluronic® block copolymers, PF68 etc); etc. These agents can also be combined with salts of the acids, e.g., sodium citrate with citric acid, to produce a buffer system. Other agents that can be used to alter the pH of the microenvironment on dissolution include salts of inorganic acids and magnesium hydroxide.

As used herein, “preservative” refers to a compound that can be added to an antibody formulation to help maintain stability of the antibody over time by, for example, reducing the impact of bacterial, fungal or other unwanted organic growth. The addition of a preservative may also facilitate the production of a multi-use (multiple-dose) formulation. Examples of potential preservatives include, but are not limited to, octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as, for example, phenol, butyl and benzyl alcohol, alkyl parabens such as methyl or propyl paraben, catechol, resorcinol, cyclohexanol, 3-pentanol, and m-cresol.

As used herein, a “stabilizer” is a compound or compounds that act to stabilize a pharmaceutical formulation with respect to solvency, viscosity, pH, purity or other such measures of stability. Exemplary stabilizers herein include, but are not limited to, saccharides, surfactants and amino acids.

The formulations described herein are, for example, solutions including a commercial drug product to be used in a clinical setting. Formulations described herein can be specific to a particular application and/or route of administration. Formulations for therapeutic antibody drug products are specific to each antibody and are extremely important for the drug product's activity and stability.

The disclosed methods include those for reducing the viscosity of concentrated antibody formulations, primarily to enable administration via common routes of administration, e.g., subcutaneous or intravitreal (IVT) injection, where needed. The carbohydrate residues/chains present on the glycosylated form of a monoclonal IgG antibody play an important role in the solubility of the antibody. This is especially the case at high solution concentrations. Consequently, aglycosylated antibodies are less soluble and more prone to aggregation than their glycosylated counterparts. The methods described herein for the formulation of aglycosylated monoclonal therapeutic antibodies with reduced viscosity and/or reduced tendency for aggregation include one or more of the following steps:

(1) increasing the total ionic strength of the formulation through the addition of salts and/or other buffer components; or

(2) altering the pH of the formulation to be within a predetermined range, without compromising biological activity; or

(3) adding a surfactant at concentrations in the range of ˜0.01% to ˜0.4% in the final formulation; or

(4) adding one or more amino acids that lower the viscosity, and/or increase the stability, of the formulated solution. These steps can be accomplished by addition a buffer, salt, surfactant and/or amino acids to a solution of an aglycosylated antibody.

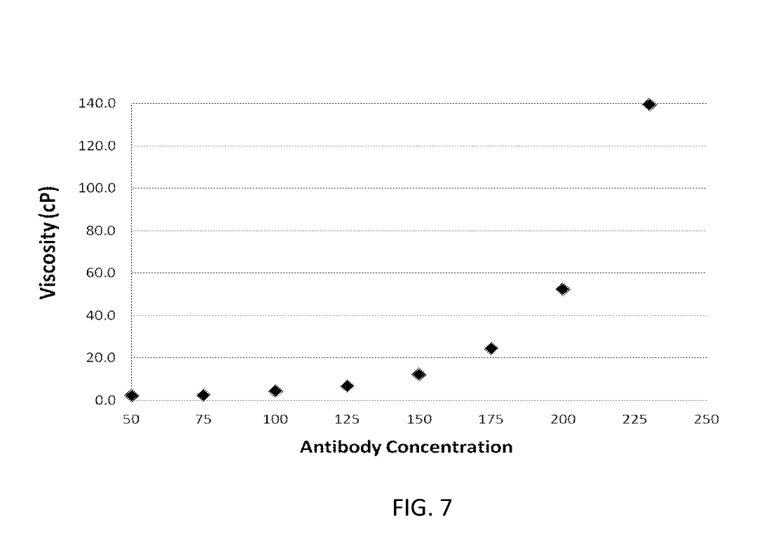

Therapeutic antibody formulations may utilize any of a variety of different buffers, excipients, salts, sugars and other additives that reduce the viscosity of the antibody solution and/or otherwise increase the solubility of the antibody in solution, and thereby prevent aggregation. Described herein are novel and unique antibody formulations suitable for a high concentration aglycosylated antibody in a stable and relatively low viscosity solution, and methods of making such formulations. Aglycosylated antibodies can be prepared in formulations described herein at concentrations of up to 200 mg/mL or higher. These formulations and methods produce, for example, a formulation for an aglycosylated antibody at a concentration of 160 mg/mL at a viscosity of only 20 cP at 22° C. (FIG. 7). The prior art provides a limited number of methods for making low concentration formulations of aglycosylated antibody, and provides no methods for high concentration formulations at low (or reduced) viscosity.

High concentration formulations are required for effective use of therapeutic antibodies via IVT administration for treatment of, for example, ocular disorders. The typical volume of a single IVT injection is limited to approximately 50 μL. To administer an effective amount of the drug via IVT injection, a high concentration formulation is usually needed. The disclosed formulations can be used, for example, for a treatment for ocular disorders wherein the antibody will be administrated through IVT injection. The disclosed formulations are also intended for treatment of systemic disorders, the treatment of which may require repeated administrations via subcutaneous injection or intravenous injection. Without the availability of a high concentration formulation, systemic administration via subcutaneous injection might not be possible or practical.

As used herein, “viscosity” refers to “kinematic viscosity” or “absolute viscosity.” “Kinematic viscosity” is a measure of the resistive flow of a fluid under the influence of gravity. Absolute Viscosity=Kinematic Viscosity×Density. Viscosity is concentration dependent. Several previously described formulations consider the highest attainable concentration of antibody to be the concentration at which the viscosity of the solution reaches 20 cP. Using the methods described herein, the viscosity of the formulations described herein reaches 20 cP at an antibody concentration of 160 mg/mL. Formulations of therapeutic IgG antibodies typically reach a viscosity of 20 cP at far lower concentrations, usually at less than 100 mg/mL. The high concentration of this formulation at a viscosity of 20 cP is one unique and advantageous property of the formulations described herein.

The formulations and methods described herein provide for high concentration antibody formulations, e.g., solutions, including, for example, high concentration aglycosylated antibody formulations. The antibody concentration can range from, for example, about 100 mg/mL to about 200 mg/mL or higher, including, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL and about 200 mg/ml or higher.

In the context of IVT injections, a therapeutic antibody formulated at 160 mg/mL could be administered via a 50 μL IVT injection to deliver up to 8 mg of antibody per eye. For subcutaneous (SC) administration, a volume of ≦1.5 mL would provide up to 240 mg with a single injection. To provide even larger doses with each administration, even higher concentration formulations are required. The methods described herein can be used to create formulations for aglycosylated antibodies at concentrations above 160 mg/mL.

To cover the broadest range of potential applications (and routes of administration) for various types of diseases and for treatment of the greatest variety of human individuals, the disclosed methods and formulations provide a therapeutic aglycosylated antibody at concentrations of, for example, 20 mg/mL, 50 mg/mL, 160 mg/ml, and 200 mg/mL. Methods and formulations described herein can be used for any aglycosylated antibody, any hybrid antibody, and/or any and all Fab-Fc antibody constructs that are generally categorized as fusion proteins. For therapeutic applications, application of the methods and formulations described herein produces a solution of aglycosylated antibody (or functional derivative thereof) that demonstrates reduced viscosity, low to no particulate formation, no low molecular weight (LMW) impurities and/or degradation products, and no high molecular weight (HMW) molecules (aggregates). Additionally, aglycosylated antibodies formulated using the methods described herein exhibit long term stability.

The term “antibody fragment,” “antigen-binding fragment,” or similar terms are known in the art and can, for example, refer to a fragment of an antibody that retains the ability to bind to a target antigen to, for example, inhibit the activity of the target antigen. Such fragments include, e.g., a single chain antibody, a single chain Fv fragment (scFv), an Fd fragment, a Fab fragment, a Fab′ fragment or a F(ab′)2 fragment A scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods and formulations described herein (Todorovska, A. et al., J. Immunol. Methods, 248:47-66, 2001; Hudson, P. & Kortt, J., J. Immunol. Methods, 231:177-89, 1999; Poljak, R., Structure, 2:1121-3, 1994; Rondon, I. & Marasco, W., Annu. Rev. Microbiol., 51:257-83, 1997). An antigen-binding fragment can also include the variable region of a heavy chain polypeptide and the variable region of a light chain polypeptide. An antigen-binding fragment can thus include the CDRs of the light chain and heavy chain polypeptide of an antibody. The term “antibody fragment” also can include, e.g., single domain antibodies such as camelized single domain antibodies (Muyldermans, S. et al., Trends Biochem. Sci., 26:230-5, 2001; Nuttall, S. et al., Curr. Pharm. Biotech., 1:253-63, 2000; Reichmann, L. & Muyldermas, S., J. Immunol. Meth., 231:25-38, 1999; PCT Publication Nos. WO 94/04678 and WO 94/25591; and U.S. Pat. No. 6,005,079, the entire contents of each of which are herein incorporated by reference). The term “antibody fragment” also includes single domain antibodies including two V_{H} domains with modifications such that single domain antibodies are formed.

Evaluation of amino acids, buffers, excipients, sugars, surfactants and pH value adjustments are the factors that can be manipulated and controlled in the development of antibody formulation. Addition of one or more amino acids generally has the effect of reducing intermolecular interactions, which generally serves to reduce aggregation at high concentrations. Surfactants increase antibody solubility. Excipients tend to affect the molecular diameter of the antibody, which also affects aggregation and solubility. The addition of buffers, salts, and/or sugars, as well as manipulation of solution pH, all have various effects on solution viscosity and the tendency of the solution towards forming aggregate antibody molecules.

Stable solutions with low viscosity, which are free of small particles, are less likely to form antibody aggregates. Such solutions are generally considered to be the result of good formulations. Such solution properties are crucial for antibody formulation at higher concentrations (and especially concentrations above 100 mg/mL).

### Choice of Buffer

Possible buffers include acetate, citrate, succinate, histidine or phosphate. An additional buffer composition included for use in the formulations described herein is the histidine-arginine buffer. Examples of histidine buffers include histidine chloride and arginine chloride, histidine acetate and arginine acetate, histidine phosphate and arginine phosphate, histidine sulfate and arginine sulfate, histidine succinate and arginine succinate, etc. The specific buffer identified in several of the examples provided herein is histidine acetate and arginine acetate.

The histidine-arginine buffer is prepared by titrating L-histidine (free base, solid) with acetic acid (liquid) and by titrating L-arginine (free base, solid) with acetic acid (liquid). In various embodiments, the histidine-arginine buffer is prepared at any pH within the range of 4.5 through 6.5. In one embodiment, the histidine-arginine buffer has a pH of 5.5. The histidine-arginine buffer can be histidine succinate and arginine succinate. The histidine succinate and arginine succinate buffer may be prepared at any pH along the range of pH values between 4.5 to 6.5 and coming at increments of 0.1. In one particular embodiment, the histidine succinate and arginine succinate buffer has a pH of 5.5. In another embodiment, the buffer has a pH of 6.0.

### Choice of Salt as Excipient

Salts can act as excipients in formulations of therapeutic antibodies. In the formulations described herein, the salt used in either the lower or high concentration formulations disclosed here can be any one from the group of; sodium chloride, sodium thiocyanate, ammonium thiocyanate, ammonium sulfate, ammonium chloride, calcium chloride, zinc chloride and sodium acetate. Addition of at least 150 mM of NaCl helps to keep the molecular diameter of the antibody at a minimum.

### Choice of Surfactant

Depending on the specific needs of the formulation and the antibody, the methods of formulation may include addition of a surfactant if needed to increase solubility of the antibody. Examples of surfactants include, but are not limited to, polysorbate-80 (PS-80) added at 0.01% to 5%. Sugars such as sucrose, trehalose, and mannitol can also be used and may be present in an amount ranging from approximately 0.05% to 5%.

### Formulations at Low Vs. High pH Values

Formulations and methods of producing formulations are described for a stable formulation for high-concentration antibody solutions with reduced viscosity at concentrations ranging from 50 to ˜200 mg/mL. At the highest concentration, ˜200 mg/mL, formulation at a lower pH and with the appropriate additives as described in the examples, the solution viscosity can be reduced to less than or equal to 50 cp. In one embodiment, described herein is a method for reducing the viscosity of a solution at ˜200 mg/mL by altering the pH to be lowered to ≦5.5 or elevated to >6, wherein the kinematic viscosity is reduced from approximately 100 cP to approximately 50 cP or less. In a particular aspect, the pH is any tenth pH value within the applicable pH range. For example, for preparation of a lower pH formulation, the specific pH value may be chosen from any of the values consisting of 4.0, 4.1, 4.2, etc. through 5.5. At the higher pH range, example values range, at increments of 0.1, from 6.1 through 12.

### Addition of Amino Acids

In a specific aspect, the amino acids are chosen from the group consisting of arginine, glycine, lysine, histidine, glutamic acid, aspartic acid, isoleucine, leucine, alanine, phenylalanine, tyrosine, tryptophan, methionine, serine, and proline. The amino acids histidine and arginine can be used for formulations of aglycosylated antibodies. Addition of arginine was found to lower viscosity. Addition of methionine was found to reduce methionine oxidation, which provides stability to the formulation.

### Reconstitution Formulation

Described herein is a reconstituted formulation wherein a lyoprotectant, such a sugar, is also added. In a particular aspect, the lyoprotectant sugar can be, for example, sucrose or trehalose, and may be present in an amount ranging from 60 to 300 mM. In one embodiment, the formulation can be reconstituted with a diluent containing a buffer or salt. The protein concentration in the reconstituted formulation typically ranges from 2 to 40 times the pre-lyophilization concentration.

### Customization

Formulations may be customized to a specific type of antibody or even to a specific antibody sequence. However, some formulations may be of broader utility and can be used for any one of several different antibodies, for solutions prepared at low or high protein concentrations. The formulation methods disclosed herein were designed for aglycosylated therapeutic antibodies. However, these methods may also be used for other large biologic proteins (>100 kD) formulated for therapeutic use.

Following extensive testing of many potential combinations of known buffers, excipients, stabilizers, and other additives and conditions, the following exemplary formulations have been developed:

(1) 20 mg/mL in 50 mM Histidine, 110 mM NaCl, 2% sucrose, and 0.01% PS-80, pH 6.5;

(2) 50 mg/mL in 20 mM Histidine, 150 mM NaCl, 0.02% PS-80 pH 6.0;

(3) 160 mg/mL in 20 mM Histidine, 150 mM NaCl, 0.02% PS-80, pH 6.0; and

(4) 200 mg/mL in 20 mM Histidine, 150 mM NaCl, 0.02% PS-80, pH 6.0.

The first formulation, at 20 mg/mL is preferably for IV administration. For the three high concentration formulations (2 through 4 above), the optimal concentration of NaCl was determined to be between ˜110 and ˜150 mM with 0.01% to 0.02% polysorbate (PS-80). Addition of various excipients and other buffer combinations did not reduce the viscosity of solution formulations. These formulations were essentially particle free, low viscosity and provided homogenous solutions even upon dilution in saline. Additions of arginine and methionine are expected to further reduce the viscosity of the high concentration formulations. A pH of 6.0 was determined to be a preferred environment for formulations for both IVT and subcutaneous (SC) administrations. Concentrations ranging from 50-200 mg/mL of an aglycosylated antibody were achieved with the high concentration formulation methods described herein. Typically, therapeutic antibody formulations are considered feasible for subcutaneous administration (are “syringeable”) at viscosities of less than or equal to approximately 50 cp. At 50 mg/mL the viscosity of the evaluated aglycosylated antibody was ˜2 cP, whereas the viscosity of the solution at 200 mg/mL was found to be 50 cP.

The buffers for formulation of an aglycosylated antibody, according to the methods described herein, generally produce a solution that is buffered within a pH range within ˜4.0 and ˜8.0. For example, the buffer used may maintain the solution at a pH range between ˜5.0 and ˜7.0. A histidine buffer can be used to maintain pH between 5.8 and 6.2. Examples of buffers that will control the pH in the appropriate range include acetate, succinate, gluconate, histidine, citrate, glycylglycine, and other organic acid buffers. The histidine buffer can be histidine-acetate or histidine-HCl. In another embodiment, the histidine buffer is maintained between pH 5.5 and 6.5.

An amino acid can be used as a stabilizer. The amino acid employed may be in the L-form. Examples of amino acids that can be included as stabilizers in the preparations and/or formulations herein include: histidine, arginine, glycine, phenylalanine, aspartic acid, glutamic acid, lysine, asparagine, and/or alanine.

An example of a preservative useful for the described formulations is benzyl alcohol.

A surfactant can also be used in the formulations described herein. The surfactant can be, for example, polysorbate 20 or polysorbate 80. Other surfactants may be included to prevent or reduce aggregation or denaturation of the monoclonal antibody in the preparation and/or formulation.

Exemplary diluents include sterile water, bacteriostatic water for injection (BWFI), a pH buffered solution (e.g., phosphate-buffered saline), sterile saline solution, Ringer's solution or dextrose solution. In an alternative embodiment, diluents can include aqueous solutions of salts and/or buffers.

For IV administration at low concentration, a saccharide may be added to the formulation. Examples of saccharides that may be used include glucose, sucrose, trehalose, lactose, fructose, maltose, dextran, glycerin, dextran, erythritol, glycerol, arabitol, sylitol, sorbitol, mannitol, melibiose, melezitose, raffinose, mannotriose, stachyose, maltose, lactulose, maltulose, glucitol, maltitol, lactitol, isomaltulose, etc. The formulations described herein can include a non-reducing disaccharide, such as trehalose or sucrose. Sugar alcohols include, but are not limited to, monoglycosides, maltose, lactulose and maltulose. The glycosidic side group can be either glucosidic or galactosidic. A lyoprotectant can be the non-reducing sugars trehalose or sucrose.

The formulations described herein are suitable for administration via a bolus or continuous infusion over a period of time, by intramuscular, intraperitoneal, intracerobrospinal, subcutaneous, intra-articular, intrasynovial, intrathecal, oral, topical, or inhalation routes. The formulations described herein also enable administration via intravitreal administration. The formulations can be administered via subcutaneous (i.e., beneath the skin) injection. Such injections include, for example, those that are self-administered. They also include pre-filled syringes, disposable syringes, and other products that provide various methods for subcutaneous injection. Other devices that may be used in conjunction with the formulations provided herein include Inject-ease™ and Genject™ devices, injector pens (such as the GenPen™), needleless devices (e.g., MediJector™ and BioJector™), and subcutaneous patch delivery systems.

### Stability Testing

The stability and quality of the monoclonal antibody formulation may be assessed by the size distribution (polydispersity) of the antibody, the % Area (Size Exclusion Chromatography) of monomer protein, at optical density 280 (OD₂₈₀), or by particle size distribution, aggregation, and/or fragmentation. Accelerated stability is conducted at a temperature of 40° C. Size distribution can be assessed, for example, using size exclusion chromatography-high performance liquid chromatography (SEC-HPLC). The percentage monomer loss (as measured by SEC-HPLC) over time is measured at an accelerated temperature of 40° C.

### Articles of Manufacture

The articles of manufacture include containers for storing antibody solutions, bottles, vials (e.g., dual chamber vials), syringes (such as dual chamber syringes), tubes, and other devices and materials commonly used for storing and administering medications in liquid suspension. The container holding the antibody solution may be formed from a variety of materials such as glass or plastic. The container holding the formulation may be a multi-use vial, which allows for repeat administrations (e.g., from 2-6 administrations) of the high concentration formulation. The article of manufacture may further include other materials desirable from a commercial and user standpoint, including other buffers, diluents, filters, needles, syringes, and package inserts with instructions for use.

### EXAMPLES

### Example 1: Excipient Screening in Various Buffer Systems

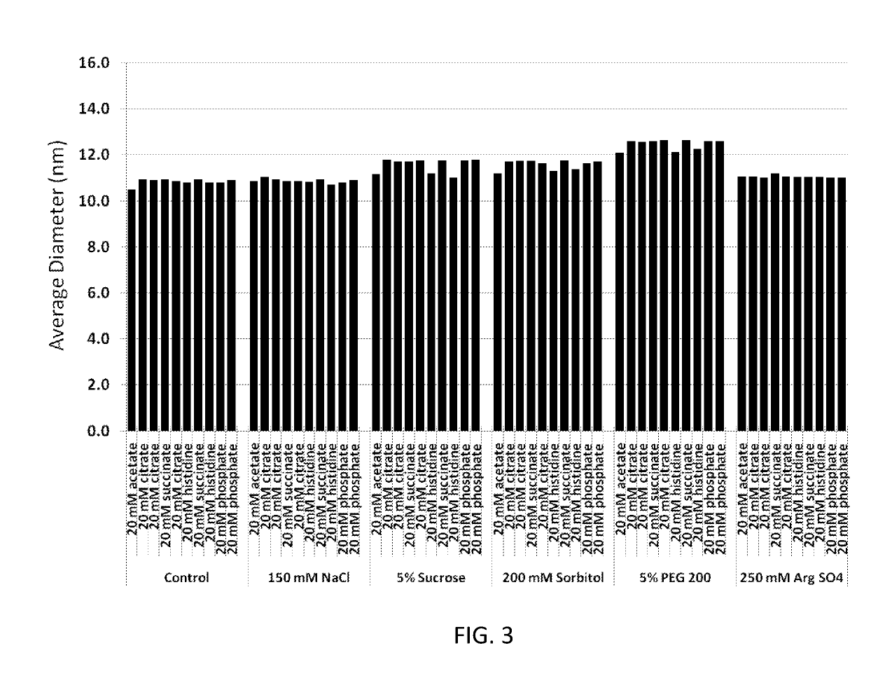

Using formulations of the Test Antibody, 20 mM of each of the acetate (pH 5.0), citrate (pH 5.0, 5.5, and 6.0), succinate (pH 5.5 and 6.0), histidine (pH 6.0 and 6.5), and phosphate (pH 7.0 and 7.5) buffers were evaluated in combination with each of the following excipients; 150 mM NaCl, 5% sucrose, 200 mM Sorbitol, 250 mM arginine sulfate, and 5% Poly Ethylene Glycol (PEG 200). Monodisperse size distribution was observed for controls, 150 mM NaCl, Sorbitol, and PEG. Polydispersity was observed for sucrose and arginine. Results are shown in FIG. 3.

### Example 2: Buffer Screening

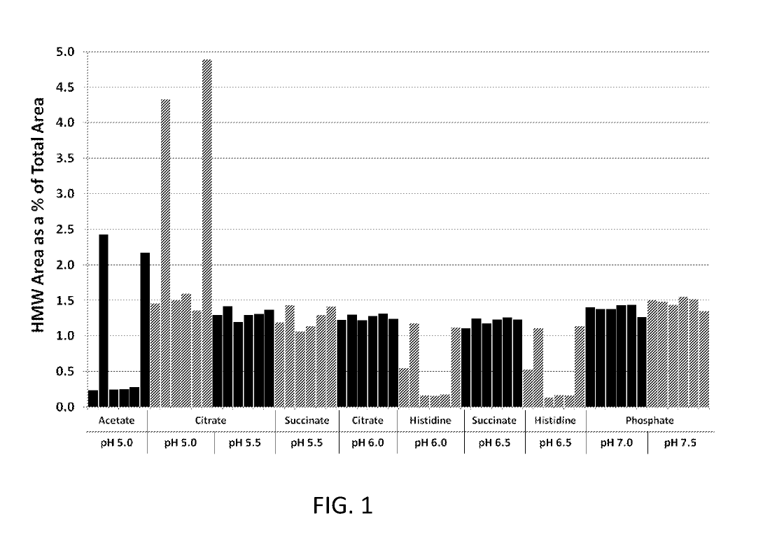

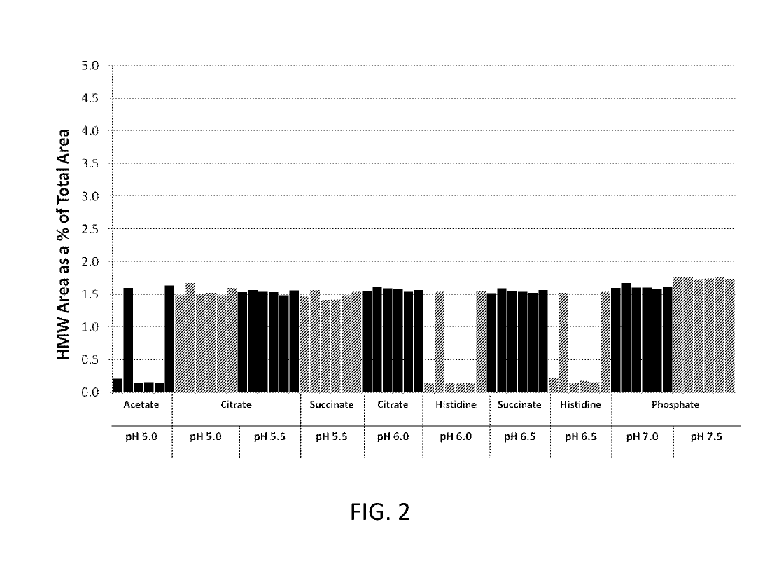

Under baseline biophysical screening at 5° C., acetate (pH 5.0), histidine (pH 6.0) and histidine (pH 6.5) showed greater than 99% monomer peak compared to other buffers such as citrate (pH 5.0, 5.5 and 6.0), succinate (pH 5.5 and 6.0), and phosphate (pH 7.0 and 7.5) where the monomer peak was 98% with an additional peak for nearly 1.9%. Therefore, formulation with 99% peak area were taken for consideration. Results are shown in FIGS. 1 and 2.

### Example 3: Evaluation of Formulations with Various Concentrations of PS-80

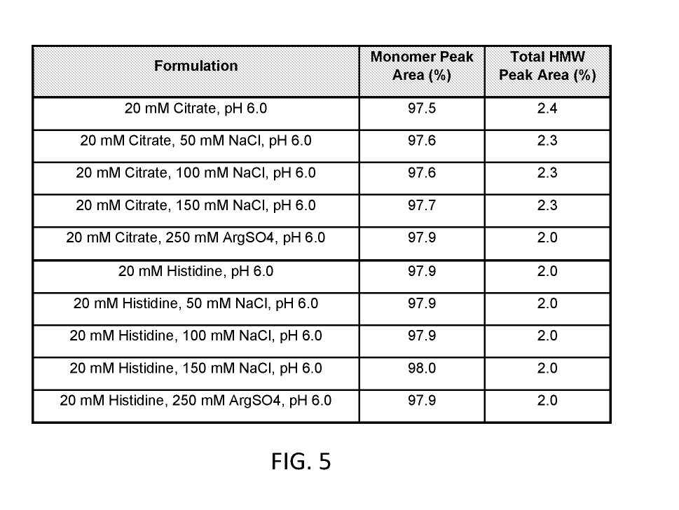

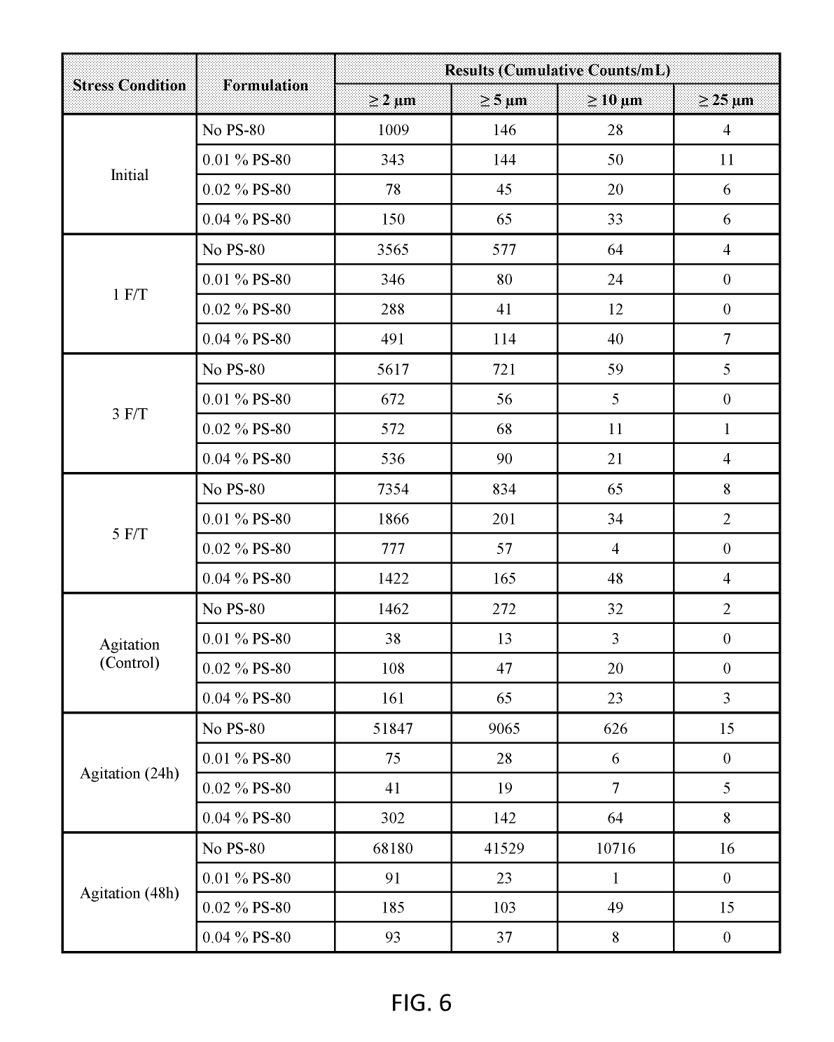

Formulations of the Test Antibody with various concentrations of PS-80 (0, 0.01, 0.02, and 0.04%) were evaluated under stress. Data demonstrate that addition of 0.01 to 0.02% PS-80 provides additional stability to the formulated Test Antibody. Results are shown in FIGS. 5 and 6.

### Example 4: Evaluation of pH Change with Addition of NaCl

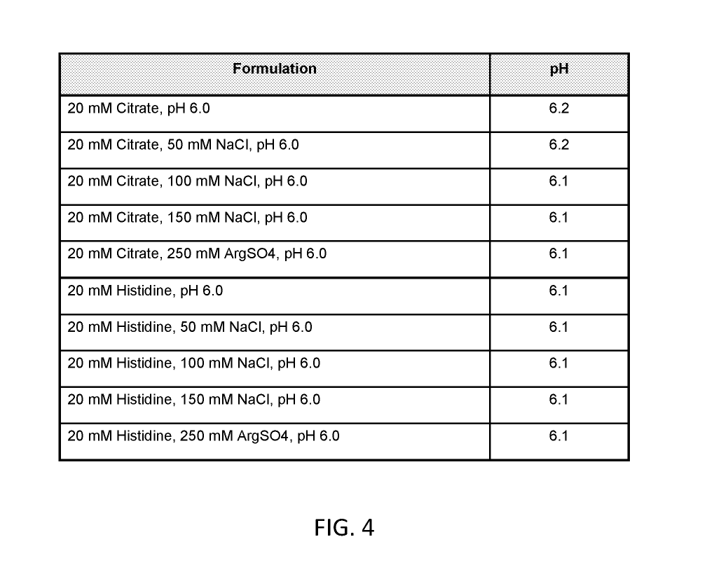

Concentrations of NaCl ranging from 0 to 150 mM were evaluated in both citrate and histidine buffers. Results shown in FIG. 4 demonstrate that at all levels of NaCl evaluated, the tested formulations using citrate or histidine buffer maintained a pH in the range of 6.0 to 6.2.

### Example 5: SEC Evaluation of Monomer Peak Area with Addition of NaCl

Concentrations of NaCl ranging from 0 to 150 mM were evaluated in both citrate and histidine buffers. When evaluated using size exclusion chromatography, the monomer peak area was found to be highest where 150 mM NaCl was added. The majority of buffer combinations gave an area greater than 97.5 to 97.9. Results are shown in FIG. 5.

### Example 6: High Concentration Formulations with Citrate Vs. Histidine Buffer

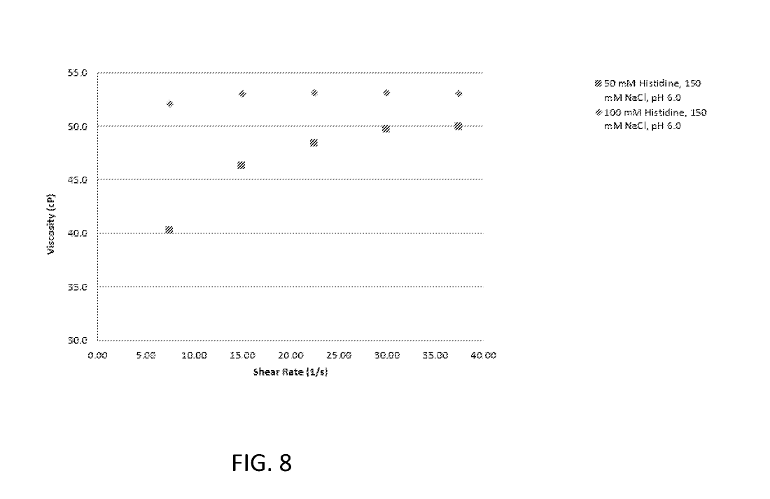

Two high-concentration formulations of Test Antibody (225 mg/mL and 230 mg/mL), which each include 150 mM of NaCl, were directly compared. The 225 mg/mL formulation used 20 mM citrate while the 230 mg/mL formulation used 20 mM histidine. Results are shown in FIG. 8.

### Example 7: Formulations for IV Administration

20 mg/mL of Test Antibody in 50 mM histidine, 110 mM NaCl, 2% sucrose, and 0.01% PS-80, pH 6.5 or, 50 mg/mL of Test Antibody in 50 mM histidine, 110 mM NaCl, 2% sucrose, and 0.01% PS-80, pH 6.5.

### Example 8: High Concentration Formulations for Administration Via Intravitreal or Subcutaneous Injection

160 mg/mL of Test Antibody in 20 mM histidine, 150 mM NaCl, 0.02% PS-80, pH 6.0 or, 200 mg/mL of Test Antibody in 20 mM histidine, 150 mM NaCl, 0.02% PS-80, pH 6.0, and arginine and methionine.

### Example 9: Effect of Test Antibody Concentration on Mean Viscosity at 22° C.

Concentration s of Test Antibody at 50, 75, 100, 125, 150, 175, and 200 mg/mL were evaluated in 20 mM histidine, 150 mM sodium chloride at pH 6.0 at 22° C. Mean viscosity increased with increasing concentration. Viscosity was ˜52 cP at 200 mg/mL and ˜20 cP at 160 mg/mL. Viscosity would be further decreased with the addition of arginine and methionine. Results are shown in FIG. 7.

### Example 10: High-Concentration Antibody Formulations and Viscosity Characteristics

High-concentration antibody formulations were tested for stability and viscosity. The formulations contained antibody concentrations as shown in the below tables. Data for FIGS. 8-12 are provided in the following tables.

TABLE 1
Data for FIG. 8.

| | Concentration | Temperature | Speed | Torque | Viscosity |
|---|---|---|---|---|---|
| Formulation | (mg/mL) | (° C.) | (RPM) | (%) | (cP) |
| 50 mM | 209 | 22° C. | 1.0 | 13.4 | 40.2 |
| Histidine, | | | 2.0 | 30.5 | 46.3 |
| 150 mM | | | 3.0 | 47.9 | 48.4 |
| NaCl, | | | 4.0 | 65.1 | 49.7 |
| pH 6.0 | | | 5.0 | 82.1 | 49.9 |
| 100 mM | 219 | 22° C. | 1.0 | 17.2 | 52.1 |
| Histidine, | | | 2.0 | 34.9 | 53.0 |
| 150 mM | | | 3.0 | 51.4 | 53.1 |
| NaCl, | | | 4.0 | 69.0 | 53.1 |
| pH 6.0 | | | 5.0 | 86.4 | 53.0 |

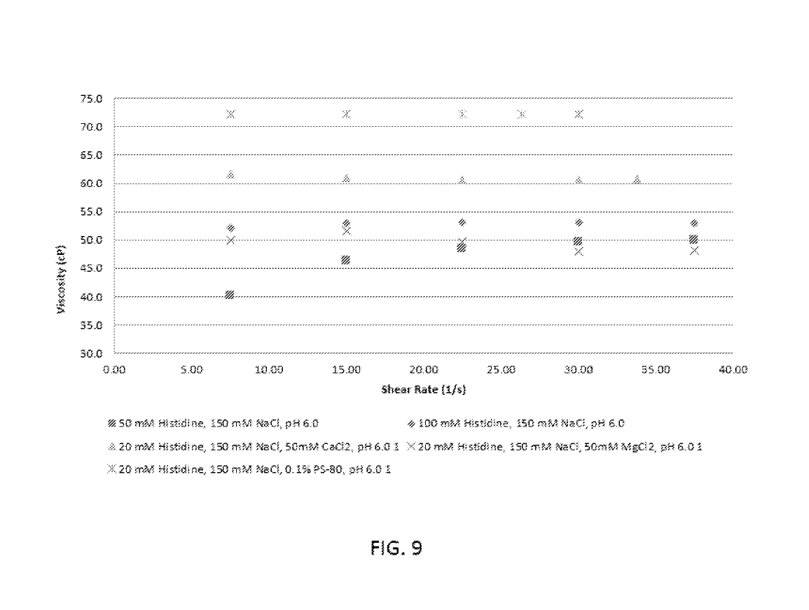

TABLE 2
Data for FIG. 9.

| | | Temper- | | | |
|---|---|---|---|---|---|
| | Concentration | ature | Speed | Torque | Viscosity |
| Formulation | (mg/mL) | (° C.) | (RPM) | (%) | (cP) |
| 50 mM | 209 | 22° C. | 1.0 | 13.4 | 40.2 |
| Histidine, | | | 2.0 | 30.5 | 46.3 |
| 150 mM NaCl, | | | 3.0 | 47.9 | 48.4 |
| pH 6.0 | | | 4.0 | 65.1 | 49.7 |
| | | | 5.0 | 82.1 | 49.9 |
| 100 mM | 219 | 22° C. | 1.0 | 17.2 | 52.1 |
| Histidine, | | | 2.0 | 34.9 | 53.0 |
| 150 mM NaCl, | | | 3.0 | 51.4 | 53.1 |
| pH 6.0 | | | 4.0 | 69.0 | 53.1 |
| | | | 5.0 | 86.4 | 53.0 |
| 20 mM | 200 | 22° C. | 1.0 | 20.4 | 61.6 |
| Histidine, | | | 2.0 | 40.3 | 61.0 |
| 150 mM NaCl, | | | 3.0 | 59.5 | 60.6 |
| 50 mM CaCl₂, | | | 4.0 | 79.2 | 60.6 |
| pH 6.0 ¹ | | | 4.5 | 88.7 | 60.8 |
| 20 mM | 200 | 22° C. | 1.0 | 16.2 | 50.0 |
| Histidine, | | | 2.0 | 33.6 | 51.6 |
| 150 mM NaCl, | | | 3.0 | 49.1 | 49.7 |
| 50 mM MgCl₂, | | | 4.0 | 63.1 | 48.0 |
| pH 6.0 ¹ | | | 5.0 | 77.3 | 48.1 |
| 20 mM | 200 | 22° C. | 1.0 | 23.5 | 72.3 |
| Histidine, | | | 2.0 | 47.3 | 72.2 |
| 150 mM NaCl, | | | 3.0 | 70.8 | 72.3 |
| 0.1% PS-80, | | | 3.5 | 82.6 | 72.3 |
| pH 6.0 ¹ | | | 4.0 | 94.7 | 72.3 |

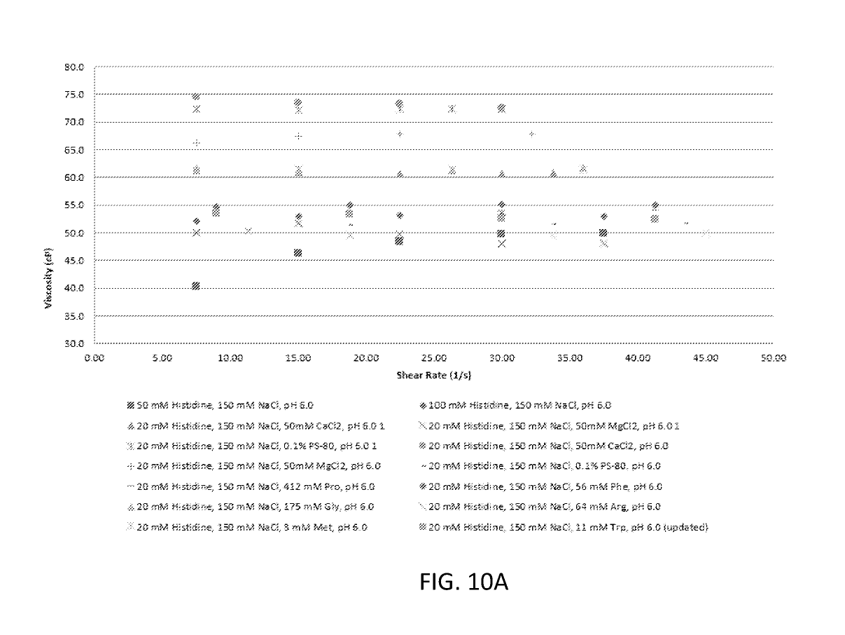

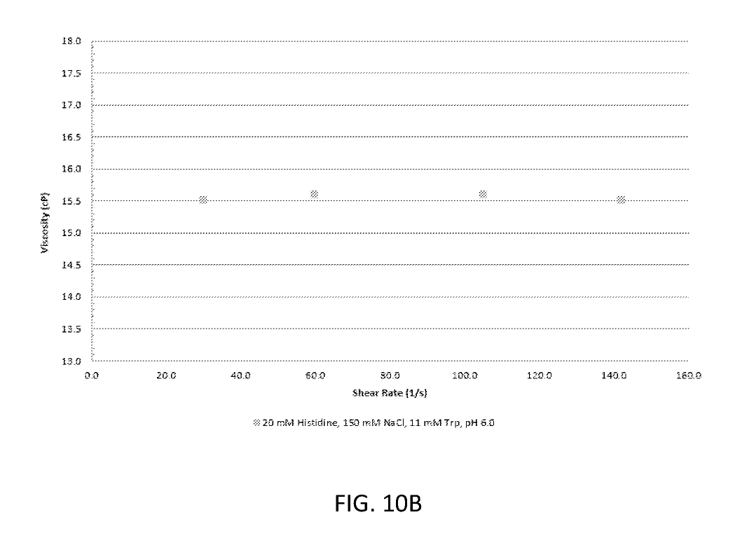

TABLE 3
Data for FIG. 10

| | | Temper- | | | |
|---|---|---|---|---|---|
| | Concentration | ature | Speed | Torque | Viscosity |
| Formulation | (mg/mL) | (° C.) | (RPM) | (%) | (cP) |
| 20 mM | 214 | 22° C. | 1.2 | 21.0 | 53.6 |
| Histidine, | | | 2.5 | 43.6 | 53.7 |
| 150 mM NaCl, | | | 4.0 | 70.6 | 53.9 |
| 412 mM Pro, | | | 5.5 | 97.2 | 54.0 |
| pH 6.0 | | | | | |
| 20 mM | 213 | 22° C. | 1.2 | 21.5 | 54.7 |
| Histidine, | | | 2.5 | 44.6 | 54.9 |
| 150 mM NaCl, | | | 4.0 | 71.8 | 55.1 |
| 56 mM Phe, | | | 5.5 | 98.6 | 55.0 |
| pH 6.0 | | | | | |
| 20 mM | 165 | 22° C. | 4.0 | 20.2 | 15.5 |
| Histidine, | | | 8.0 | 40.5 | 15.6 |
| 150 mM NaCl, | | | 14.0 | 71.0 | 15.6 |
| 11 mM Trp, | | | 19.0 | 96.1 | 15.5 |
| pH 6.0 | | | | | |
| 20 mM | 210 | 22° C. | 1.2 | 20.8 | 53.4 |
| Histidine, | | | 2.5 | 43.6 | 53.3 |
| 150 mM NaCl, | | | 4.0 | 68.5 | 52.6 |
| 11 mM Trp, | | | 5.5 | 93.5 | 52.4 |
| pH 6.0 | | | | | |
| (updated) | | | | | |
| 20 mM | 207 | 22° C. | 1.0 | 20.0 | 61.3 |
| Histidine, | | | 2.0 | 40.0 | 61.1 |
| 150 mM NaCl, | | | 3.5 | 70.4 | 61.3 |
| 175 mM Gly, | | | 4.8 | 96.6 | 61.7 |
| pH 6.0 | | | | | |
| 20 mM | 205 | 22° C. | 1.5 | 24.6 | 50.3 |
| Histidine, | | | 2.5 | 40.4 | 49.5 |
| 50 mM NaCl, | | | 4.5 | 72.6 | 49.6 |
| 64 mM Arg, | | | 6.0 | 97.0 | 49.8 |
| pH 6.0 | | | | | |
| 20 mM | 211 | 22° C. | 1.0 | 20.0 | 61.3 |
| Histidine, | | | 2.0 | 40.1 | 61.5 |
| 150 mM NaCl, | | | 3.5 | 69.8 | 61.4 |
| 3 mM Met, | | | 4.8 | 96.4 | 61.4 |
| pH 6.0 | | | | | |

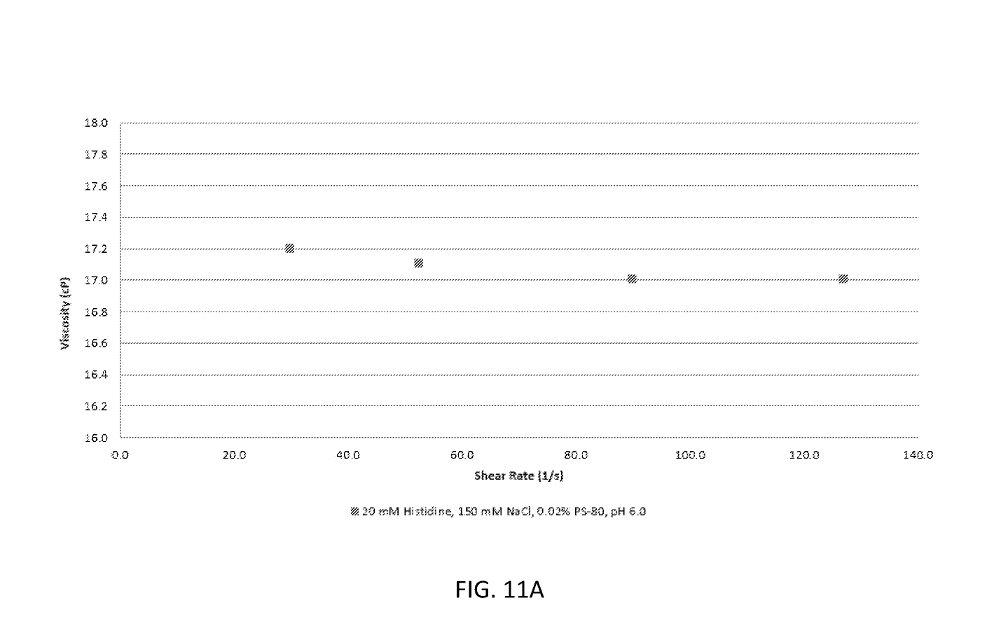

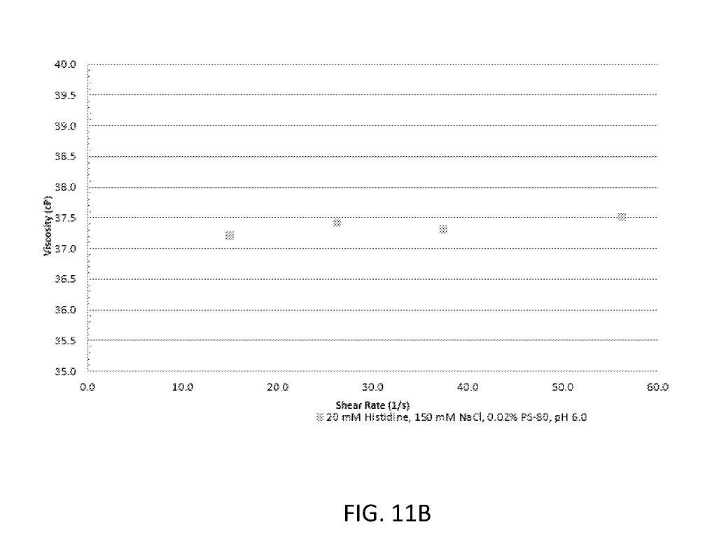

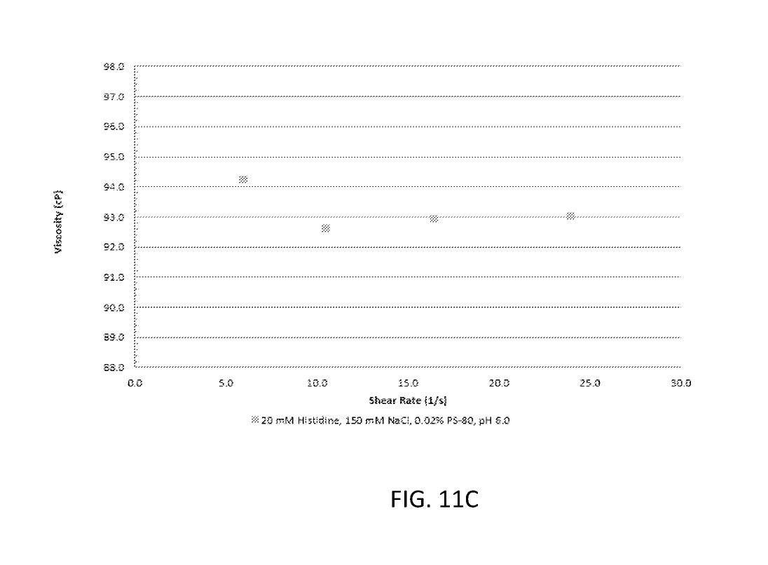

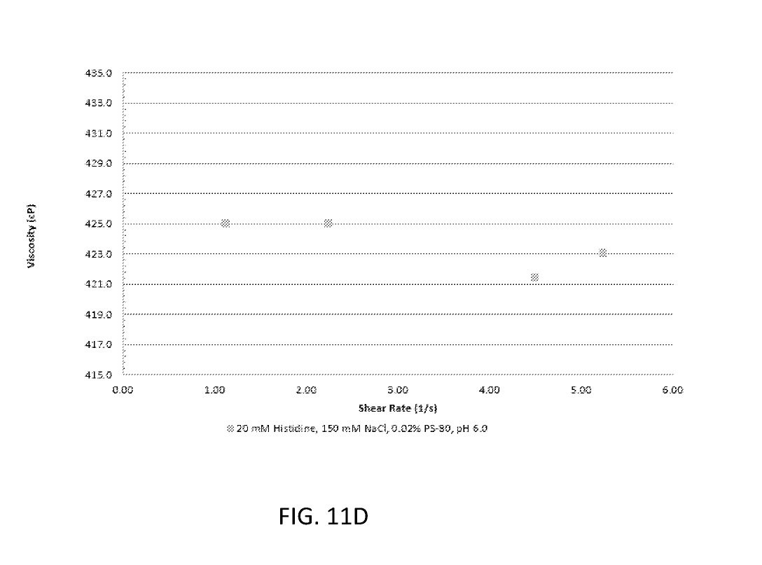

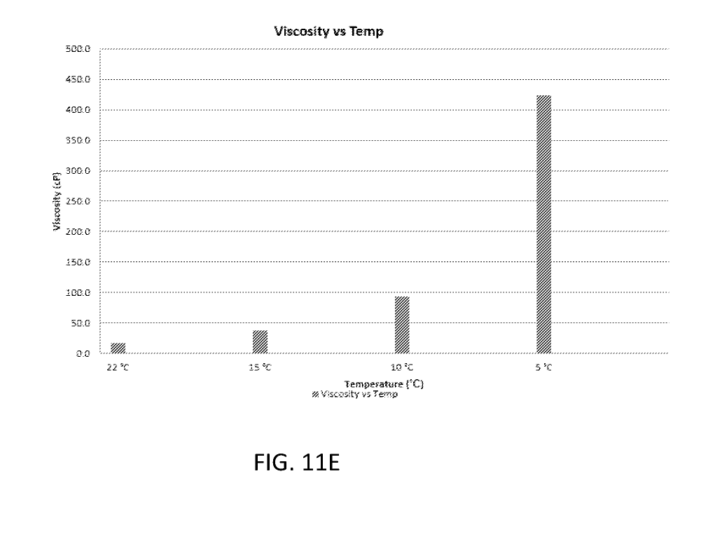

TABLE 4
Data for FIG. 11.

| | | Temper- | | | |
|---|---|---|---|---|---|
| | Concentration | ature | Speed | Torque | Viscosity |
| Formulation | (mg/mL) | (° C.) | (RPM) | (%) | (cP) |
| 20 mM | 162 | 22° C. | 4.0 | 22.4 | 17.2 |
| Histidine, | | | 7.0 | 39.0 | 17.1 |
| 150 mM NaCl, | | | 12.0 | 66.5 | 17.0 |
| 0.02% PS-80, | | | 17.0 | 94.5 | 17.0 |
| pH 6.0 | | | | | |
| 20 mM | 162 | 15° C. | 2.0 | 24.5 | 37.2 |
| Histidine, | | | 3.5 | 42.6 | 37.4 |
| 150 mM NaCl, | | | 5.0 | 61.2 | 37.3 |
| 0.02% PS-80, | | | 7.5 | 91.9 | 37.5 |
| pH 6.0 | | | | | |
| 20 mM | 162 | 10° C. | 0.8 | 24.6 | 94.2 |
| Histidine, | | | 1.4 | 42.4 | 92.6 |
| 150 mM NaCl, | | | 2.2 | 66.6 | 92.9 |
| 0.02% PS-80, | | | 3.2 | 96.7 | 93.0 |
| pH 6.0 | | | | | |
| 20 mM | 162 | 5° C. | 0.15 | 20.8 | 425.0 |
| Histidine, | | | 0.30 | 41.6 | 425.0 |
| 150 mM NaCl, | | | 0.60 | 82.6 | 421.4 |
| 0.02% PS-80, | | | 0.70 | 96.6 | 423.0 |
| pH 6.0 | | | | | |

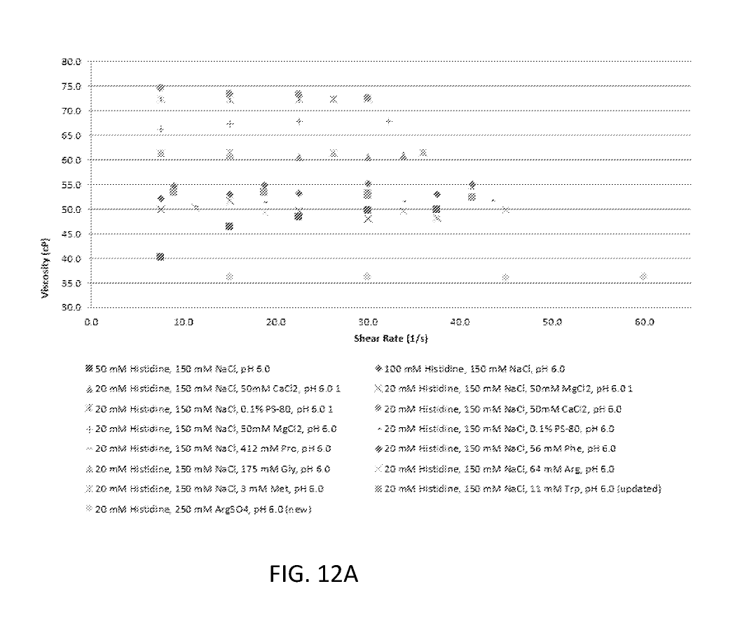

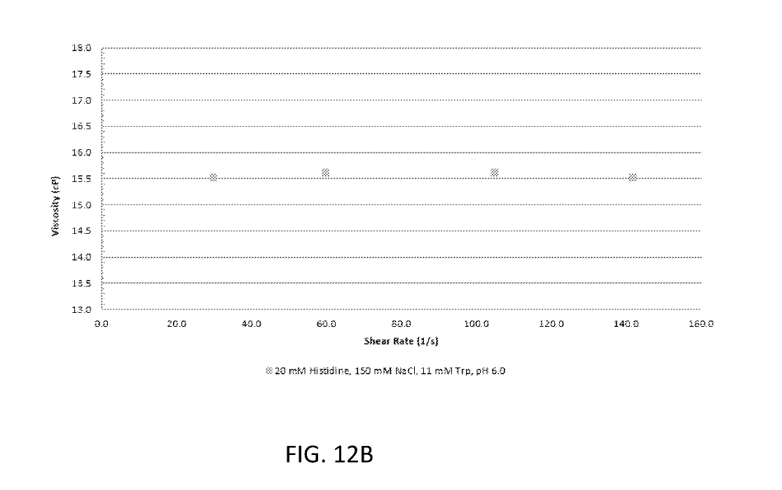

TABLE 5
Data for FIG. 12.

| | | Temper- | | | |
|---|---|---|---|---|---|
| | Concentration | ature | Speed | Torque | Viscosity |
| Formulation | (mg/mL) | (° C.) | (RPM) | (%) | (cP) |
| 50 mM | 209 | 22 ° C. | 1.0 | 13.4 | 40.2 |
| Histidine, | | | 2.0 | 30.5 | 46.3 |
| 150 mM NaCl, | | | 3.0 | 47.9 | 48.4 |
| pH 6.0 | | | 4.0 | 65.1 | 49.7 |
| | | | 5.0 | 82.1 | 49.9 |
| 100 mM | 219 | 22° C. | 1.0 | 17.2 | 52.1 |
| Histidine, | | | 2.0 | 34.9 | 53.0 |
| 150 mM NaCl, | | | 3.0 | 51.4 | 53.1 |
| pH 6.0 | | | 4.0 | 69.0 | 53.1 |
| | | | 5.0 | 86.4 | 53.0 |
| 20 mM | 200 | 22° C. | 1.0 | 20.4 | 61.6 |
| Histidine, | | | 2.0 | 40.3 | 61.0 |
| 150 mM NaCl, | | | 3.0 | 59.5 | 60.6 |
| 50 mM CaCl₂, | | | 4.0 | 79.2 | 60.6 |
| pH 6.0 ¹ | | | 4.5 | 88.7 | 60.8 |
| 20 mM | 212 | 22° C. | 1.0 | 24.4 | 74.5 |
| Histidine, | | | 2.0 | 48.1 | 73.4 |
| 150 mM NaCl, | | | 3.0 | 71.6 | 73.2 |
| 50 mM CaCl₂, | | | 4.0 | 94.9 | 72.5 |
| pH 6.0 | | | | | |
| 20 mM | 200 | 22° C. | 1.0 | 16.2 | 50.0 |
| Histidine, | | | 2.0 | 33.6 | 51.6 |
| 150 mM NaCl, | | | 3.0 | 49.1 | 49.7 |
| 50 mM MgCl₂, | | | 4.0 | 63.1 | 48.0 |
| pH 6.0 ¹ | | | 5.0 | 77.3 | 48.1 |
| 20 mM | 207 | 22° C. | 1.0 | 21.6 | 66.2 |
| Histidine, | | | 2.0 | 44.0 | 67.4 |
| 150 mM NaCl, | | | 3.0 | 66.0 | 67.9 |
| 50 mM MgCl2, | | | 4.3 | 94.7 | 67.8 |
| pH 6.0 | | | | | |
| 20 mM | 200 | 22° C. | 1.0 | 23.5 | 72.3 |
| Histidine, | | | 2.0 | 47.3 | 72.2 |
| 150 mM NaCl, | | | 3.0 | 70.8 | 72.3 |
| 0.1% PS-80, | | | 3.5 | 82.6 | 72.3 |
| pH 6.01 | | | 4.0 | 94.7 | 72.3 |
| 20 mM | 204 | 22° C. | 1.5 | 24.7 | 50.5 |
| Histidine, | | | 2.5 | 41.7 | 51.4 |
| 150 mM NaCl, | | | 4.5 | 75.9 | 51.5 |
| 0.1% PS-80, | | | 5.8 | 97.8 | 51.7 |
| pH 6.0 | | | | | |

TABLE 6
Data for FIG. 12 (cont).

| | | Temper- | | | |
|---|---|---|---|---|---|
| | Concentration | ature | Speed | Torque | Viscosity |
| Formulation | (mg/mL) | (° C.) | (RPM) | (%) | (cP) |
| 20 mM | 214 | 22° C. | 1.2 | 21.0 | 53.6 |
| Histidine, | | | 2.5 | 43.6 | 53.7 |
| 150 mM NaCl, | | | 4.0 | 70.6 | 53.9 |
| 412 mM Pro, | | | 5.5 | 97.2 | 54.0 |
| pH 6.0 | | | | | |
| 20 mM | 213 | 22° C. | 1.2 | 21.5 | 54.7 |
| Histidine, | | | 2.5 | 44.6 | 54.9 |
| 150 mM NaCl, | | | 4.0 | 71.8 | 55.1 |
| 56 mM Phe, | | | 5.5 | 98.6 | 55.0 |
| pH 6.0 | | | | | |
| 20 mM | 165 | 22° C. | 4.0 | 20.2 | 15.5 |
| Histidine, | | | 8.0 | 40.5 | 15.6 |
| 150 mM NaCl, | | | 14.0 | 71.0 | 15.6 |
| 11 mM Trp, | | | 19.0 | 96.1 | 15.5 |
| pH 6.0 | | | | | |
| 20 mM | 210 | 22° C. | 1.2 | 20.8 | 53.4 |
| Histidine, | | | 2.5 | 43.6 | 53.3 |
| 150 mM NaCl, | | | 4.0 | 68.5 | 52.6 |
| 11 mM Trp, | | | 5.5 | 93.5 | 52.4 |
| pH 6.0 | | | | | |
| (updated) | | | | | |
| 20 mM | 207 | 22° C. | 1.0 | 20.0 | 61.3 |
| Histidine, | | | 2.0 | 40.0 | 61.1 |
| 150 mM NaCl, | | | 3.5 | 70.4 | 61.3 |
| 175 mM Gly, | | | 4.8 | 96.6 | 61.7 |
| pH 6.0 | | | | | |
| 20 mM | 205 | 22° C. | 1.5 | 24.6 | 50.3 |
| Histidine, | | | 2.5 | 40.4 | 49.5 |
| 150 mM NaCl, | | | 4.5 | 72.6 | 49.6 |
| 64 mM Arg, | | | 6.0 | 97.0 | 49.8 |
| pH 6.0 | | | | | |
| 20 mM | 201 | 22° C. | 2.0 | 21.7 | 36.2 |
| Histidine, | | | 4.0 | 47.3 | 36.2 |
| 250 mM | | | 6.0 | 70.0 | 36.0 |
| ArgSO₄, pH 6.0 | | | 8.0 | 93.8 | 36.1 |
| (new) | | | | | |
| 20 mM | 211 | 22° C. | 1.0 | 20.0 | 61.3 |
| Histidine, | | | 2.0 | 40.1 | 61.5 |
| 150 mM NaCl, | | | 3.5 | 69.8 | 61.4 |
| 3 mM Met, | | | 4.8 | 96.4 | 61.4 |
| pH 6.0 | | | | | |

### Other Embodiments

It is understood that while embodiments have been described in conjunction with the detailed description, the foregoing description is intended to illustrate and not limit the scope, which is defined by the scope of the appended claims. The materials, methods, and examples are illustrative only and not intended to be limiting. All publications, patent applications, patents, sequences, database entries and other references cited and described herein are incorporated by reference in their entireties. Other aspects, advantages and modifications are within the scope of the following claims.

What is claimed is:

1. An antibody formulation comprising a therapeutic antibody at a concentration of at least about 20 mg/mL.

2. The formulation of claim 1, wherein the therapeutic antibody concentration is at least about 100 mg/mL to about 250 mg/mL or higher.

3. The formulation of claim 2, wherein the therapeutic antibody concentration is about 160 mg/mL.

4. The formulation of claim 1, wherein the formulation is a solution with a viscosity of less than about 60 cP.

5. The formulation of claim 4, wherein the solution has a viscosity of between about 20 cP and about 50 cP.

6. The formulation of claim 4, wherein the solution has a viscosity of about 20 cP.

7. The formulation of claim 1, further comprising a buffer selected from the group consisting of: histidine buffer, citrate buffer, acetate buffer, succinate buffer, and phosphate buffer.

8. The formulation of claim 7, wherein the buffer is a histidine buffer comprising a combination selected from the group consisting of: histidine chloride and arginine chloride; histidine acetate and arginine acetate; histidine phosphate and arginine phosphate; histidine sulfate and arginine sulfate; and histidine succinate and arginine succinate.

9. The formulation of claim 1, further comprising a viscosity reducing agent selected from the group consisting of: arginine, glycine, lysine, histidine, glutamic acid, aspartic acid, isoleucine, leucine, alanine, phenylalanine, tyrosine, tryptophan, methionine, serine and proline.

10. The formulation of claim 1, further comprising an agent to prevent antibody aggregation, wherein the agent is selected from the group consisting of: arginine, glycine, lysine, histidine, glutamic acid, aspartic acid, isoleucine, leucine, alanine, phenylalanine, tyrosine, tryptophan, methionine, serine, and proline.

11. The formulation of claim 1, wherein the pH of the formulation is between about 5.0 and about 6.5.

12. The formulation of claim 1, further comprising polysorbate 80 (PS-80) at a concentration between about 0.005% and about 0.10%.

13. The formulation of claim 12, wherein said PS-80 is at a concentration of about 0.01% to about 0.04%.

14. The formulation of claim 1, further comprising salt selected from the group consisting of: sodium chloride, sodium thiocyanate, ammonium thiocyanate, ammonium sulfate, ammonium chloride, calcium chloride, arginine hydrochloride, zinc chloride and sodium acetate.

15. The formulation of claim 14, comprising sodium chloride at a concentration between about 20 mM and about 150 mM.

16. The formulation of claim 14, comprising sodium chloride at a concentration between about 150 mM and about 250 mM.

17. The formulation of claim 1, further comprising a lyoprotectant.

18. The formulation of claim 1, further comprising a sugar selected from the group consisting of: glucose, sucrose, trehalose, lactose, fructose, maltose, dextran, glycerin, dextran, erythritol, glycerol, arabitol, sylitol, sorbitol, mannitol, melibiose, melezitose, raffinose, mannotriose, stachyose, maltose, lactulose, maltulose, glucitol, maltitol, lactitol and isomaltulose.

19. The formulation of claim 18, wherein the sugar is present in an amount of about 0.5% to about 5%.

20. The formulation of claim 1, further comprising a surfactant.

21. The formulation of claim 20, wherein the surfactant is selected from the group consisting of: polysorbate 20; polysorbate 80; a poloxamer; poloxamer 188; Triton; sodium dodecyl sulfate (SDS); sodium laurel sulfate; sodium octyl glycoside; lauryl-, myristyl-, linoleyl-, and stearyl-sulfobetaine; lauryl-, myristyl-, linoleyl- and stearyl-sarcosine; linoleyl-, myristyl- and cetyl-betaine; lauroamidopropyl-, cocamidopropyl-, linoleamidopropyl-, myristamidopropyl-, palmidopropyl- and isostearamidopropyl-betaine; lauroamidopropyl; myristamidopropyl-, palmidopropyl- and isostearamidopropyl-dimethylamine; sodium methyl cocoyl- and disodium methyl oleyl-taurate; the MONAQUAT™ series; polyethyl glycol, polypropyl glycol, and copolymers of ethylene and propylene glycol; and Pluronic® block polymers.

22. The formulation of claim 1, wherein the therapeutic antibody is reconstituted from a lyophilized form.

23. The formulation of claim 22, wherein the antibody concentration in the reconstituted formulation is approximately 2-40 times greater than the protein concentration of the antibody prior to lyophilization.

24. The formulation of claim 1, wherein the antibody is selected from the group consisting of: an aglycosylated antibody, a hybrid antibody of IgG1/IgG2, a hybrid antibody IgG2/IgG4, an IgG1 antibody, an IgG2 antibody and combinations thereof.

25. The formulation of claim 24, wherein the antibody is an aglycosylated antibody.

26. The formulation of claim 1, wherein the antibody is selected from the group consisting of: an anti-properdin antibody, an anti-factor B antibody, an anti-C3 antibody, an anti-factor D antibody, an anti-factor C5 antibody, and an anti-C5b-9 antibody.

27. The formulation of claim 1, comprising 25 mM histidine, 2% sucrose, 110 mM NaCl, and 0.01% Tween at a pH about 6.5.

28. A method of administering a therapeutic antibody to a patient in need thereof comprising administering a therapeutically effective amount of a formulation of any of claims 1-27.

29. The method of claim 28, wherein the formulation is administered subcutaneously, intravitreally or intravenously.
